(19) United States
(12) Patent Application Publication
(10) Pub. No.: US 2018/0026924 A1
(43) Pub. Date: Jan. 25, 2018

(54) APPARATUS AND METHOD FOR OFFERING ANSWER LIST INFORMATION CORRESPONDING TO STRING

(71) Applicant: Samsung Electronics Co., Ltd., Gyeonggi-do (KR)

(72) Inventors: Jong Hyun HAN, Seoul (KR); Da Hye KIM, Seoul (KR); Bo Keun KIM, Gyeonggi-do (KR)

(21) Appl. No.: 15/642,695

(22) Filed: Jul. 6, 2017

(30) Foreign Application Priority Data
Jul. 19, 2016 (KR) 10-2016-0091594

(51) Int. Cl. H04L 12/58; A61B 5/024; H04L 29/08

(52) U.S. Cl. CPC H04L 51/046; H04L 67/18; A61B 5/024

(57) ABSTRACT

An electronic device and method are disclosed herein. The electronic device includes a display, a memory storing biometric information and function information, and a processor. The processor implements the method, including detecting whether a received textual message includes a keyword, when the received textual message includes a predesignated keyword, retrieve particular bio-information corresponding to the keyword from the stored bio-information, and a particular function information from the stored function information, generating one or more textual responses to the received textual message based on the retrieved particular bio-information and the particular function information, and controlling the display to display an answer list including the generated one or more textual responses.

### CLAIM OF PRIORITY

This application claims the benefit under 35 U.S.C. §119(a) of a Korean patent application filed on Jul. 19, 2016 in the Korean Intellectual Property Office and assigned Serial number 10-2016-0091594, the entire disclosure of which is hereby incorporated by reference.

### TECHNICAL FIELD

Embodiments disclosed in the present disclosure relate to technologies of offering answer list information.

### BACKGROUND

As a mobile communication technology develops, electronic devices having a display, such as wearable devices, smart phones, or the like, have spread. Such electronic devices may execute various functions such as capturing photos or videos, playing back music or videos, playing games, Internet browsing and the like. In addition, the electronic device may receive various characters through the display and may output a received message.

When a conventional wearable device receives a message, a user may input answer information into a display of a wearable device. In addition, when a conventional smart phone receives a message, a user may personally input answer information into a display of the smart phone.

For example, the user may input characters into the wearable device and the smart phone by the using an on-screen keyboard. The on-screen keyboard employs an input scheme allowing the user to personally input a character into the display of the electronic device without a physical keyboard.

However, if user characters are input into the display of the wearable device or the smart phone, the procedure of inputting answer information to a received message may be inconvenient. For example, since the wearable device may have a rather small display, clear viewing is not guaranteed. In addition, since the size of each character key is small and the physical boundary between character keys is either small or nonexistent, typographical errors may frequently occur. In addition, a user, who is unfamiliar with directly inputting a character on the display may have difficult inputting the characters into the display.

Accordingly, a user may wish to use an electronic device which automatically offers a list of possible answers to a received message. If the answer list for the received messages is automatically offered, it becomes unnecessary for the user to individually input a reply to the received messages, the convenience of the user may be increased.

Further, during the procedure of offering the answer list information, it is necessary to combine information on a body status of a user with information on the function of an electronic device. If the information on the body status of the user is combined with the information on the function of the electronic device, the answer list information, which reflects the present situation of the user, may be offered. If the answer list information, which reflects the present situation of the user, is provided, the user may input answer information to the received message by a single touch.

### SUMMARY

Aspects of the present disclosure are to address at least the above-mentioned problems and to provide at least the benefits described below. Accordingly, various embodiments of the present disclosure provide apparatuses and methods of offering at least one of answer list information when a string is received to the electronic device

According to an embodiment disclosed in the present disclosure, an electronic device is disclosed including a display, a memory configured to store bio-information of a user collected over a designated time period, and function information for executable functions of the electronic device, and a processor operating to when a textual message includes a predesignated keyword is received, retrieve particular bio-information corresponding to the keyword from the stored bio-information, and a particular function information corresponding to the retrieved bio-information from the stored function information, generate one or more textual responses to the received textual message based on the retrieved particular bio-information and the particular function information, and control the display to display an answer list including the generated one or more textual responses.

In addition, according to an embodiment disclosed in the present disclosure, an electronic device is disclosed including a display, a memory configured to store bio-information of a user recorded over a designated time period, and a processor operating to: detect whether a keyword is included in a received textual message, if the keyword is included, retrieve particular bio-information from the stored bio-information corresponding to the keyword, and generate one or more textual responses to the received textual message based on the retrieved particular bio-information, and control the display to display an answer list including the generated one or more textual responses.

Further, according to an embodiment disclosed in the present disclosure, a non-transitory storage medium may store computer-readable instructions that, when is executed by an electronic device, cause the electronic device to store bio-information of a user for a designated time period, and function information for executable functions of the electronic device, when a textual message includes a predesignated keyword is received, retrieve particular bio-information corresponding to the keyword from the stored bio-information, and a particular function information corresponding to the retrieved bio-information from the stored function information, generate one or more textual responses to the received textual message based on the retrieved particular bio-information and the particular function information, and control a display to display an answer list including the generated one or more textual responses. Other aspects, and salient features of the disclosure will become apparent to those skilled in the art from the following detailed description, which, taken in conjunction with the annexed drawings, discloses various embodiments of the present disclosure.

### BRIEF DESCRIPTION OF THE DRAWINGS

The above and other aspects and features of certain embodiments of the present disclosure will be more apparent from the following description taken in conjunction with the accompanying drawings, in which:

FIG. 1 illustrates one example of an operating environment of an electronic device, according to an embodiment;

FIG. 2 illustrates an electronic device receiving a string from a terminal, according to an embodiment;

FIG. 3 is a table illustrating bio-information, according to an embodiment;

FIG. 4 is a table illustrating function execution information, according to an embodiment;

FIG. 5 illustrates the generation of answer list information related to a period, according to an embodiment;

FIG. 6 illustrates the display of answer information on an input window, according to an embodiment;

FIG. 7 illustrates the display of answer information on a chat window, according to an embodiment;

FIG. 8 illustrates the generation of answer list information related to foods, according to an embodiment;

FIG. 9 illustrates the generation of answer list information related to a schedule, according to an embodiment;

FIG. 10 illustrates the generation of answer list information related to a user interest, according to an embodiment;

FIG. 11 illustrates the generation of answer list information related to a position, according to an embodiment;

FIG. 12 illustrates the generation of answer list information based on user input information, according to an embodiment;

FIG. 13 is a flowchart illustrating the generation of answer list information, according to an embodiment;

FIG. 14 illustrates one example of an operating environment of an electronic device, according to another embodiment;

FIG. 15 illustrates an electronic device receiving a string from a terminal, according to another embodiment; and

FIG. 16 illustrates the output of answer list information and answer information through an electronic device, according to another embodiment.

Throughout the drawings, it should be noted that like reference numbers are used to depict the same or similar elements, features, and structures.

### DETAILED DESCRIPTION

Hereinafter, various embodiments of the present disclosure may be described with reference to accompanying drawings. Accordingly, those of ordinary skill in the art will recognize that modification, equivalent, and/or alternative on the various embodiments described herein can be variously made without departing from the present disclosure. With regard to description of drawings, similar elements may be marked by similar reference numerals.

In this disclosure, the expressions “have”, “may have”, “include” and “comprise”, or “may include” and “may comprise” used herein indicate existence of corresponding features (e.g., elements such as numeric values, functions, operations, or components) but do not exclude presence of additional features.

In this disclosure, the expressions “A or B”, “at least one of A or/and B”, or “one or more of A or/and B”, and the like may include any and all combinations of one or more of the associated listed items. For example, the term “A or B”, “at least one of A and B”, or “at least one of A or B” may refer to all of the case (1) where at least one A is included, the case (2) where at least one B is included, or the case (3) where both of at least one A and at least one B are included.

The terms, such as “first”, “second”, and the like used in this disclosure may be used to refer to various elements regardless of the order and/or the priority and to distinguish the relevant elements from other elements, but do not limit the elements. For example, “a first user device” and “a second user device” indicate different user devices regardless of the order or priority. For example, without departing the scope of the present disclosure, a first element may be referred to as a second element, and similarly, a second element may be referred to as a first element.

It will be understood that when an element (e.g., a first element) is referred to as being “(operatively or communicatively) coupled with/to” or “connected to” another element (e.g., a second element), it may be directly coupled with/to or connected to the other element or an intervening element (e.g., a third element) may be present. In contrast, when an element (e.g., a first element) is referred to as being “directly coupled with/to” or “directly connected to” another element (e.g., a second element), it should be understood that there are no intervening element (e.g., a third element).

According to the situation, the expression “configured to” used in this disclosure may be used as, for example, the expression “suitable for”, “having the capacity to”, “designed to”, “adapted to”, “made to”, or “capable of”. The term “configured to” must not mean only “specifically designed to” in hardware. Instead, the expression “a device configured to” may mean that the device is “capable of” operating together with another device or other components. For example, a “processor configured to (or set to) perform A, B, and C” may mean a dedicated processor (e.g., an embedded processor) for performing a corresponding operation or a generic-purpose processor (e.g., a central processing unit (CPU) or an application processor) which performs corresponding operations by executing one or more software programs which are stored in a memory device.

Terms used in this disclosure are used to describe specified embodiments and are not intended to limit the scope of the present disclosure. The terms of a singular form may include plural forms unless otherwise specified. All the terms used herein, which include technical or scientific terms, may have the same meaning that is generally understood by a person skilled in the art. It will be further understood that terms, which are defined in a dictionary and commonly used, should also be interpreted as is customary in the relevant related art and not in an idealized or overly formal unless expressly so defined in various embodiments of this disclosure. In some cases, even if terms are terms which are defined in this disclosure, they may not be interpreted to exclude embodiments of this disclosure.

An electronic device according to various embodiments of this disclosure may include at least one of, for example, smartphones, tablet personal computers (PCs), mobile phones, video telephones, electronic book readers, desktop PCs, laptop PCs, netbook computers, workstations, servers, personal digital assistants (PDAs), portable multimedia players (PMPs), Motion Picture Experts Group (MPEG-1 or MPEG-2) Audio Layer 3 (MP3) players, mobile medical devices, cameras, or wearable devices. According to various embodiments, the wearable device may include at least one of an accessory type (e.g., watches, rings, bracelets, anklets, necklaces, glasses, contact lens, or head-mounted-devices (HMDs), a fabric or garment-integrated type (e.g., an electronic apparel), a body-attached type (e.g., a skin pad or tattoos), or a bio-implantable type (e.g., an implantable circuit).

According to various embodiments, the electronic device may be a home appliance. The home appliances may include at least one of, for example, televisions (TVs), digital versatile disc (DVD) players, audios, refrigerators, air conditioners, cleaners, ovens, microwave ovens, washing machines, air cleaners, set-top boxes, home automation control panels, security control panels, TV boxes (e.g., Samsung HomeSync™, Apple TV™, or Google TV®), game consoles (e.g., Xbox™ or PlayStation™), electronic dictionaries, electronic keys, camcorders, electronic picture frames, and the like.

According to another embodiment, an electronic device may include at least one of various medical devices (e.g., various portable medical measurement devices (e.g., a blood glucose monitoring device, a heartbeat measuring device, a blood pressure measuring device, a body temperature measuring device, and the like), a magnetic resonance angiography (MRA), a magnetic resonance imaging (MRI), a computed tomography (CT), scanners, and ultrasonic devices), navigation devices, Global Navigation Satellite System (GNSS), event data recorders (EDRs), flight data recorders (FDRs), vehicle infotainment devices, electronic equipment for vessels (e.g., navigation systems and gyrocompasses), avionics, security devices, head units for vehicles, industrial or home robots, automatic teller's machines (ATMs), points of sales (POSs) of stores, or internet of things (e.g., light bulbs, various sensors, electric or gas meters, sprinkler devices, fire alarms, thermostats, street lamps, toasters, exercise equipment, hot water tanks, heaters, boilers, and the like).

According to an embodiment, the electronic device may include at least one of parts of furniture or buildings/structures, electronic boards, electronic signature receiving devices, projectors, or various measuring instruments (e.g., water meters, electricity meters, gas meters, or wave meters, and the like). According to various embodiments, the electronic device may be one of the above-described devices or a combination thereof. An electronic device according to an embodiment may be a flexible electronic device. Furthermore, an electronic device according to an embodiment of this disclosure may not be limited to the above-described electronic devices and may include other electronic devices and new electronic devices according to the development of technologies.

Hereinafter, electronic devices according to various embodiments will be described with reference to the accompanying drawings. In this disclosure, the term “user” may refer to a person who uses an electronic device or may refer to a device (e.g., an artificial intelligence electronic device) that uses the electronic device.

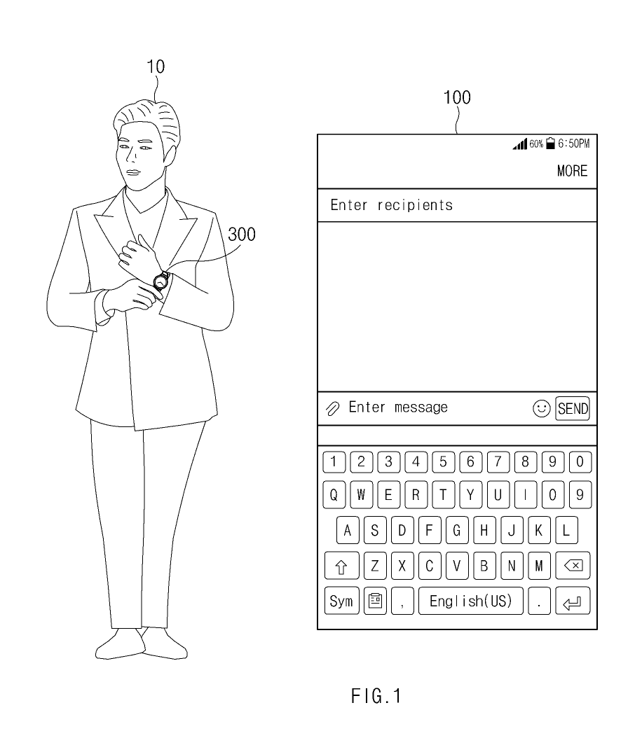

FIG. 1 illustrates one example of an operating environment of an electronic device, according to an embodiment.

Referring to FIG. 1, a biometric sensor 300 may be a device which measures information (body status information) on a body status of a user 10. For example, the biometric sensor 300 may be a blood pressure gauge, a hear rate monitor, or a weighing scale. The biometric sensor 300 may measure oxygen saturation, a caffeine level, or other such information from the blood of the user 10 without necessarily making contact with the body of the user 10. In addition, the biometric sensor 300 may measure a weight or a height of the user 10 by using an ultrasonic wave, a laser, or other such implementations.

After the biometric sensor 300 measures body status information, the biometric sensor 300 may transmit the body status information to an electronic device 100. The biometric sensor 300 and the electronic device 100 may transceive various information through bidirectional communication (e.g., Wi-Fi, ZigBee, BT/BLE), but the communication scheme between the biometric sensor 300 and the electronic device 100 are not limited thereto.

According to an embodiment, the biometric sensor 300 may be included in the electronic device 100 to measure the body status information. In the case that the electronic device 100 includes the biometric sensor 300, if the user 10 makes contact with the electronic device 100, the biometric sensor 300 may measure the blood or the heart rate of the user 10, or the like. If the user 10 injects the blood of the user 10 into the electronic device 100, the biometric sensor 300 may measure oxygen saturation, a caffeine level, or the like from the blood of the user 10.

The body status information measured by the biometric sensor 300 may be stored in a memory of the electronic device 100. The memory may store the body status information for a designated period, and the designated period may be set by the user 10. For example, if the user 10 sets the designated period to one week, the memory may store the body status information for one week.

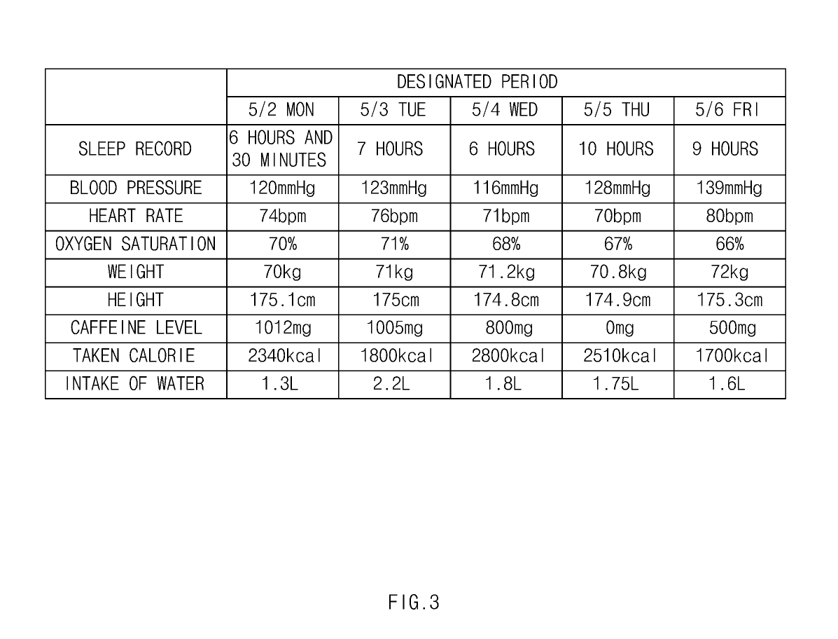

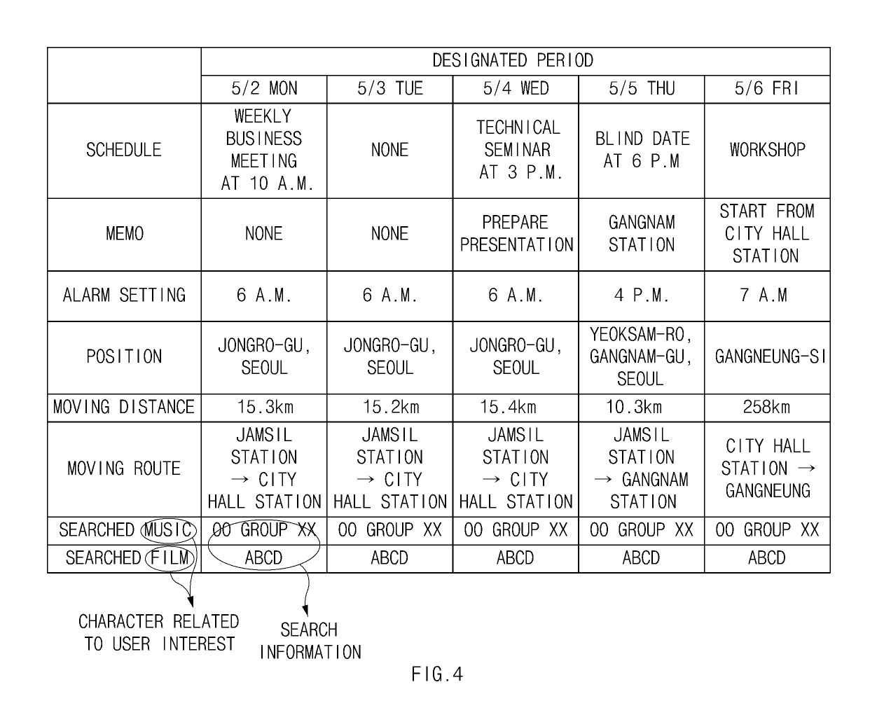

The electronic device 100 may generate at least one message reply (e.g., a piece of answer list information, or a list of possible responses) based on the bio-information and function execution information (or function information). The bio-information may be information obtained by accumulating the body status information measured by the biometric sensor 300 for the designated period, which will be described with reference to FIG. 3. The body status information may be information representing the body status of the user 10. The body status information may include, for example, the sleep time, the blood pressure, the heart rate, the oxygen saturation, the weight, the height, the caffeine level, the taken calories, or the intake of water of the user 10, or the like. The function execution information, which serves as information related to the function of the electronic device 100, may include the schedule, the memo, the moving distance, the location, or the moving route of the user 10, information searched for through the electronic device 100, or the like. The function execution information may be stored in the memory together with the bio-information, and the detailed embodiment of the function execution information will be described with reference to FIG. 4 below.

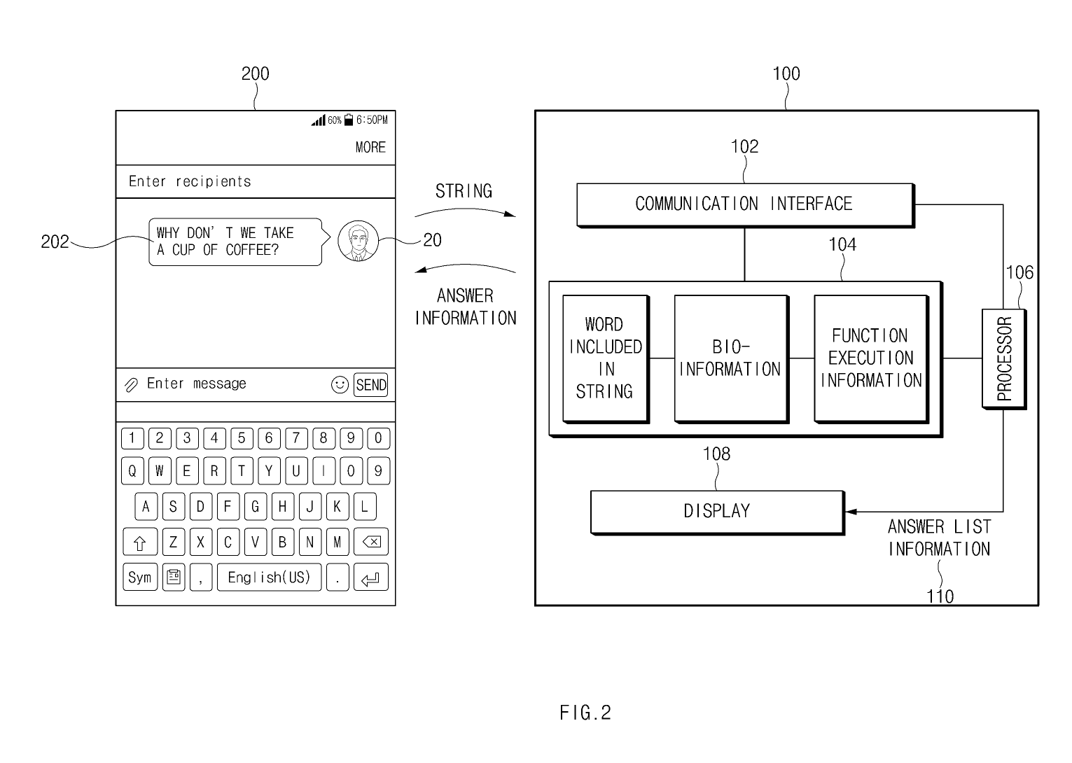

FIG. 2 illustrates the electronic device 100 receiving a string 202 (e.g., a message comprising a string of characters) from a terminal 200, according to an embodiment. FIG. 3 is a table illustrating bio-information, according to an embodiment, and FIG. 4 is a table illustrating function execution information, according to an embodiment. Hereinafter, the electronic device 100 according to an embodiment will be described with reference to FIGS. 2 to 4.

Referring to FIG. 2, according to an embodiment of the present disclosure, the electronic device 100 may include a communication interface 102, a memory 104, a processor 106, and a display 108.

The string 202 (or textual message) may be the combination of a series of characters (or keywords) input into the terminal 200 by a terminal user 20. The terminal 200 may be an electronic device, such as a smart phone or a tablet PC, allowing communication. The terminal 200 and the electronic device 100 may be the same type of devices, or different types of devices.

The communication interface 102 may receive the string 202 from the terminal 200 and may provide the string 202 to the processor 106. The communication interface 102 may transmit answer information to the string 202 to the terminal 200. The communication interface 102 may establish communication between the electronic device 100 and the terminal 200. For example, the communication interface 102 may be connected with a network through wireless communication or wired communication to make communication with the terminal 200 or a server.

For example, the wireless communication may use at least one of Long-Term Evolution (LTE), LTE-Advanced (LTE-A), Code Division Multiple Access (CDMA), Wideband CDMA (WCDMA), Universal Mobile Telecommunications System (UMTS), Wireless Broadband (WiBro), or Global System for Mobile Communications (GSM), as a cellular communication protocol. In addition, the wireless communication may include, for example, short-range communication. The short-range communication may include, for example, at least one of Wireless Fidelity (Wi-Fi), Bluetooth, Near Field Communication (NFC), Magnetic Stripe Transmission (MST), or GNSS.

The MST may generate a pulse based on transmission data by using an electromagnetic signal, and the pulse may be used to generate a magnetic field signal. The electronic device 100 may transmit the magnetic field signal to a point of sales (POS). The POS may detect the magnetic field signal using an MST reader and may recover the data by converting the detected magnetic field signal into an electrical signal.

The GNSS may include at last one of Global Positioning System (GPS), Global Navigation Satellite System (Glonass), Beidou Navigation Satellite System (Beidou) or the European global satellite-based navigation system (Galileo) according to an area or a bandwidth that is used. Hereinafter, in the present disclosure, the terms “GPS” and “GNSS” may be interchangeably used. The wired communication may include, for example, at least one of Universal Serial Bus (USB), High Definition Multimedia Interface (HDMI), Recommended Standard-232 (RS-232) or Plain Old Telephone Service (POTS). The network may include a telecommunication network, for example, at least one of a computer network (LAN or WAN), the Internet, or a telephone network.

The memory 104 may store the bio-information and the function execution information. Referring to FIGS. 2 and 3, the bio-information may be information obtained by accumulating the body status information, which represents the body status of the user 10, for a designated period. For example, the bio-information may represent sleep records accumulated for one week, blood pressures accumulated for one month, or heart rates accumulated for one year. In addition, the bio-information may include the variation in the oxygen saturation, the weight, the height, the caffeine level, the taken calorie, or the intake of water of the user 10, or the like for the designated period (or from a specific past time point to a present time point).

The designated period may be a period specified by the user 10. For example, the designated period may be one week, one month, or one year or may be one week from a present time point, as the user 10 specifies the period. In addition, the designated period may a word indicating a period included in the string 202. For example, if the string 202 is “How was the last week?”, the designated period may be the last week.

Referring to FIGS. 2 and 4, the function execution information may be information related to the function of the electronic device 100. For example, the function execution information may include the schedule, the memo, the alarm setting, the moving distance, the location, or the moving route of the user 10, information searched for through the electronic device 100, or the like. The schedule or the memo of the user 10 may be information directly input by the user 10 through the electronic device 100, and the moving distance, the location, or the moving route of the user 10 may be information measured through the GPS in the electronic device 100. The information searched for through the electronic device 100 may be information frequently searched for by the user 10 through the Internet explorer or an application and may be stored in the memory 104.

In addition, the function execution information may be information related to the function of the electronic device 100 for the designated period. For example, the function execution information may be information on the schedules for one week, the moving distances for one month, or music or films searched for by a user for one year. The designated period may be substantially identical to the designated period described with reference to FIGS. 2 and 3. According to an embodiment of the present disclosure, the answer list information (or answer list) may be generated based on the bio-information and the function execution information within the designated period. Accordingly, the answer list information, which reflects the status of the user 10, may be provided.

Referring to FIG. 2 again, the memory 104 may store the relation between a word included in the string and the bio-information. For example, if the word “coffee” is included in the string, the memory 104 may store the connection relation between “coffee” and “caffeine level” such that the caffeine level, which is the bio-information related to the “coffee”, is searched for by the processor 106. For example, if the word “coffee” is included in the string, the memory 104 may store the connection relation between “coffee” and “sleep record” such that the “sleep record”, which is the bio-information related to the “coffee”, is searched for by the processor 106. However, the connection relation between the word included in the string and the bio-information is not limited to the above-described embodiment. For example, multiple bio-information may be searched for by the processor 106 according to words included in the string.

According to an embodiment, the memory 104 may store the connection relation between the bio-information and the function execution information. For example, if the “caffeine level”, which is the bio-information, is searched for by the processor 106, the memory 104 may store the connection relation between the “caffeine level” and the “schedule” such that the “schedule”, which is the function execution information related to the “caffeine level”, may be searched for by the processor 106. For example, if the “caffeine level”, which is the bio-information, is searched for by the processor 106, the memory 104 may store the connection relation between the “caffeine level” and the “alarm setting” such that the “alarm setting”, which is the function execution information related to the “caffeine level”, may be searched for by the processor 106. However, the connection relation between the bio-information and the function execution information is not limited to the above-described embodiment. For example, multiple function execution information may be searched for by the processor 106 according to the bio-information.

According to an embodiment, the memory 104 may include a volatile and/or a non-volatile memory 104. The memory 104 may store, for example, an instruction or data related to at least one of other elements included in the electronic device 100. According to an embodiment, the memory 104 may store software and/or a program. The program may include, for example, a kernel, middleware, an Application Programming Interface (API) and/or an application program (or application). At least a portion of the kernel, the middleware, or the API may be referred to as an operating system (OS).

If the string 202 is received, the processor 106 may extract the bio-information related to at least a portion of the string 202 from the memory 104. For example, if the string 202, which is “why don't we take a cup of coffee?” is received, the processor 106 may extract “caffeine level” related to “coffee” from the memory 104.

If the bio-information is extracted from the memory 104, the processor 106 may extract the function execution information related to the extracted bio-information from the memory 104. Since the processor 106 extracts “caffeine level” which is the bio-information as described above, the processor 106 may extract the function execution information related to the schedule from the memory 104. For example, the function execution information may be “meeting”.

If the function execution information is extracted, the processor 106 may generate at least one piece of answer list information 110 corresponding to the string 202 based on the extracted bio-information and the extracted function execution information. If the processor 106 determines that the caffeine level represents a higher level and a meeting is present before the string 202 is received as described above, the processor 106 may generate the answer list information 110 which is “I had a cup of coffee at that meeting, so I don't want to take it”.

The answer list information 110 may be information to be answer information to the string 202. If the user selects any one piece of information from at least one piece of answer list information 110, the selected answer list information may be the answer information. For example, according to the above embodiment, “Let's have a drink.” and “I have a business meeting at 2 o'clock, so let's have a drink tomorrow.” may be the answer list information 110. If the user selects “Okay, let's have a drink”, “Okay, let's have a drink” may be the answer information. In addition, if the user selects “I have a business meeting at 2 o'clock, so let's have a drink tomorrow.”, “I have a business meeting at 2 o'clock, so let's have a drink tomorrow.” may be the answer information.

According to an embodiment, the answer list information 110 may provide a plurality of words. To this end, the answer list information 110 may be provided in the form of a word list. If the answer list information 110 including a plurality of words is provided, the user may form the answer information by selecting at least one from among the words included in the answer list information 110. According to the above embodiment, the answer list information may include “drink”, “meeting”, “2 o'clock”, “business meeting”, “do not drink”, or the like. If the answer list information is provided, the user may select “2 o'clock”, “business meeting”, or “do not drink” and the answer information of “I have a business meeting at 2 o'clock, so I do not drink.” may be provided.

According to an embodiment, the processor 106 may include one of a central processing unit (CPU), an application processor (AP), or a communication processor (CP), or more. For example, the processor 106 may perform, for example, an operation or data processing for the control and/or the communication of at least one of other elements included in the electronic device 100.

In addition, the processor 106 may control a plurality of hardware elements or software elements connected with the processor 106 by driving the OS or the application. The processor 106 may perform the processing of various data and various operations. For example, the processor 106 may be implemented with a system on chip SoC. According to an embodiment, the processor 106 may further include a graphic processing unit (GPU) and/or image signal processor 106. The processor 106 may load an instruction or data, which is received from at least one of other elements (e.g., non-volatile memory), to a volatile memory and may process the instruction or the data. In addition, the processor 106 may store various data in the non-volatile memory.

The display 108 may display the answer list information. The display 108 may display the string 202, an input window, into which a user inputs a character, a chat window, on which the string is displayed, or an on-screen keyboard, as well as the answer list information. In addition, the display 108 may simultaneously display the answer list information, the string 202, the input window, the chat window, or the on-screen keyboard, or may display any one of them, according to situations.

According to an embodiment, the display 108 may include a liquid crystal display (LCD), a liquid crystal diode (LED) display, an organic LED (OLED) display, a microelectromechanical systems (MEMS) display, or an electronic paper display. The display 108 may display, for example, various contents (e.g., texts, images, videos, icons, or symbols) for a user. The display 108 may include a touch screen. For example, the display 108 may receive, for example, a touch input, a gesture input, a proximity input, or a hovering input by using an electronic pen or a part of a body of the user.

The display 108 may include a panel, a hologram device, or a projector. For example, the panel may be flexibly, transparently, or wearably implemented. The panel may be configured together with the touch panel in the form of one module. The hologram device may display a 3D image in the air by using the interference of light. The projector may display an image by projecting light on a screen. For example, the screen may be positioned inside or outside the electronic device. According to one embodiment, the display 108 may further include a control circuit to control the panel, the holograph device, or the projector.

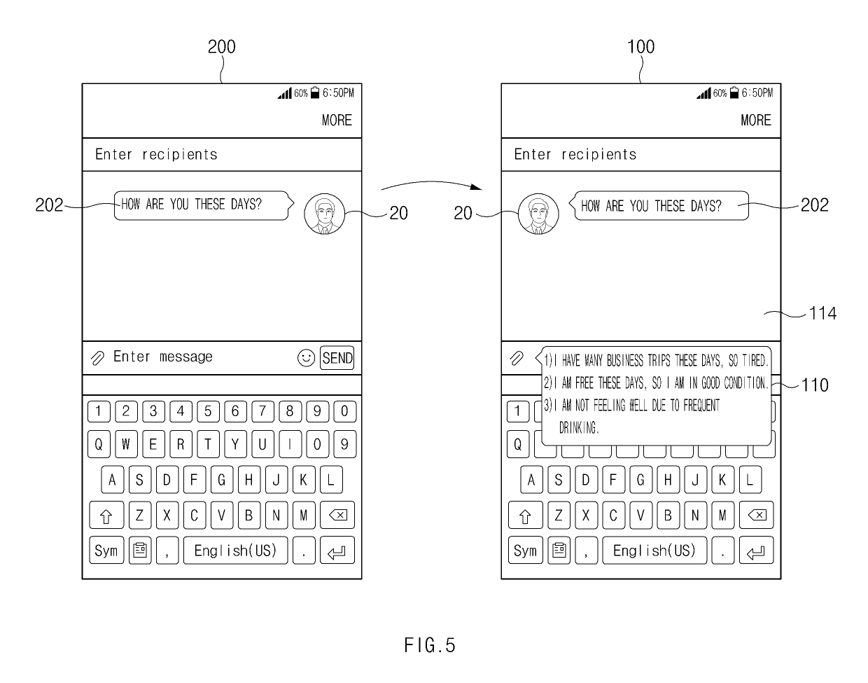

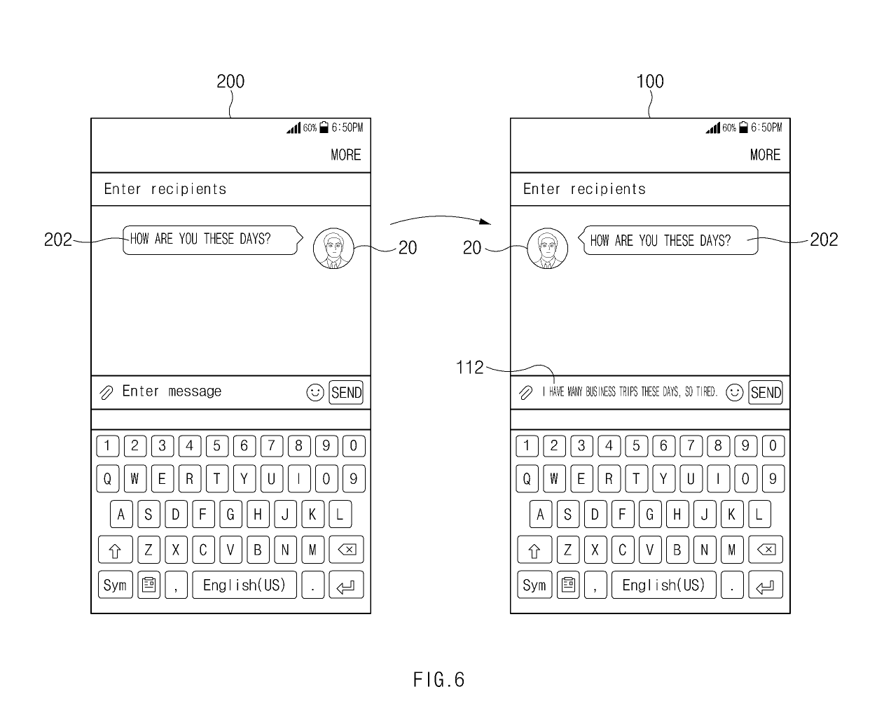

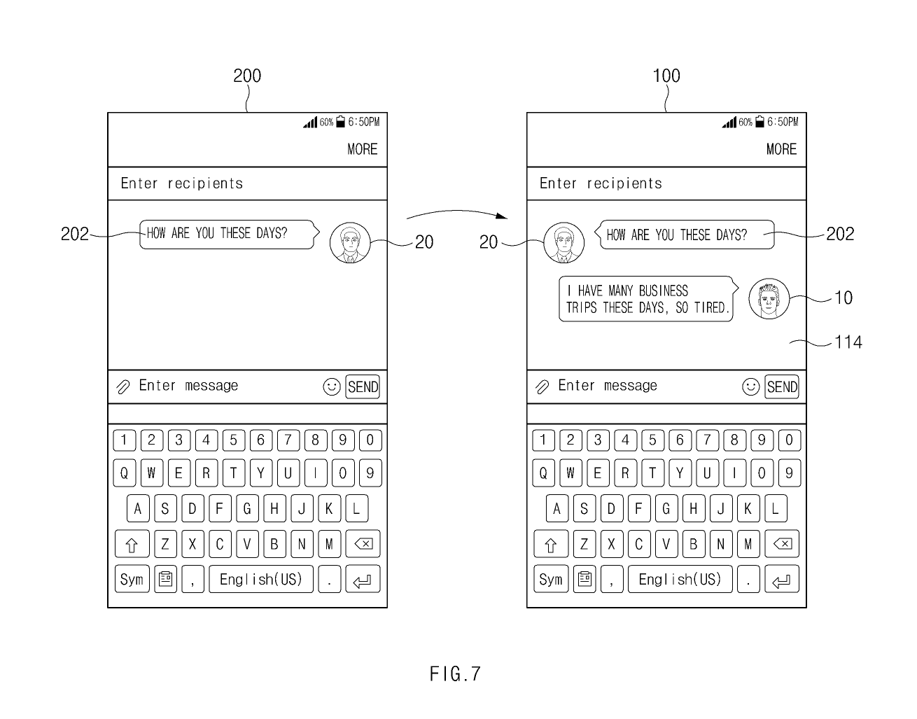

FIG. 5 illustrates the generation of answer list information 110 related to a period, according to an embodiment. FIG. 6 illustrates the display of answer information on an input window 112, according to an embodiment. FIG. 7 illustrates the display of answer information on a chat window 114, according to an embodiment. Hereinafter, the generation of the answer list information 110 according to one embodiment of the present disclosure will be described with reference to FIG. 2 and FIGS. 5 to 7.

If a terminal user 20 inputs the string 202 into the terminal 200 and transmits the string 202 to the electronic device 100, the electronic device 100 may display the received string 202 on the chat window 114. If the string 202 is received, the processor 106 may determine whether a character related to a period (e.g., a time period, a time duration, or some other related term) is in the string 202. If the processor 106 determines the character related to the period as being included in the string 202, the processor 106 may extract the character from the string 202. For example, after a character list related to the period is stored in the memory 104 inside the electronic device, if the character related to the period is present in the received string 202, the processor 106 may extract the character. Referring to FIG. 5, the characters of “these days”, which are related to the period, may be stored in the memory 104. Accordingly, if the string 202 of “How are you these days?” is received, the processor 106 may extract “these days” which are characters related to the period. If the period is not defined like “these days”, the processor 106 may randomly define the period, and the period may be one week or one month as described above.

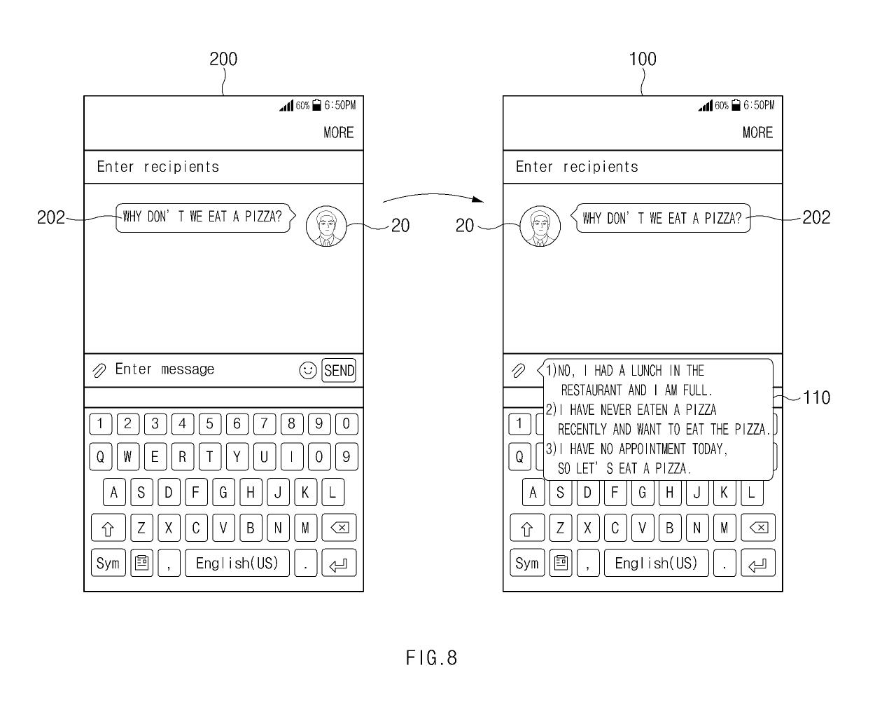

The processor 106 may determine whether the character related to the period is present while extracting another character. For example, the processor 106 may extract a character related to foods, a schedule, a user interest, a location, or the like, as well as the period. Even a character list related to foods, the schedule, the user interest, the location, or the like may be stored in the memory, and the processor 106 may extract the character related to the period, the foods, the schedule, the user interest, the location, or the like if the string is received. Hereinafter, the manners of extracting the character related to the foods, the schedule, the user interest, and the location will be described with reference to FIGS. 8, 9, 10, and 11, respectively.

Referring to FIG. 5 again, if the character related to the period is present in the string 202, the processor 106 may extract bio-information, which corresponds to (e.g., is obtained for) the time period related to the character, from the memory 104. For example, the memory 104 may store the bio-information, which is obtained for the period included in the string 202, and the function execution information obtained for the period included in the string 202. The processor 106 may extract the bio-information and the function execution information stored in the memory 104.

If the bio-information and the function execution information are extracted, the processor 106 may generate at least one response (e.g., pieces of answer list information 110) (or text response) based on the extracted bio-information and the extracted function execution information. For example, if “these days” (e.g., characters related to the period) is extracted as illustrated in FIG. 5, the processor 106 may extract sleep records and heart rates, which are accumulated during a time period indicated by the phrasing “these days,” such as data stored during a recent week or month, from the memory 104. If the bio-information is extracted, the processor 106 may determine schedules, which are performed for one week or for one month, from the function execution information.

If it is determined that the user's sleep periods have been short, or if there have been many business meetings during the time period, the processor 106 may generate responses (e.g., the answer list information 110) such as “I have many business meetings these days, so tired.” Conversely, if it is determined that the user's sleep periods have been long and there have not been any or many meetings during the time interval, the processor 106 may generate the answer list information 110 of “I am free these days, so I am in good condition.” In addition, if it is determined that a level of a particular gastrointestinal hormone is lower than an average level, and many meetings with friends had been scheduled in the interval, the processor 106 may generate the answer list information 110 of “I am not feeling well due to frequent drinking.”

The gastrointestinal hormone may be a peptide hormone distributed by a gastrointestinal tract. For example, the gastrointestinal hormone may be gastrin, secretin, motilin, or cholecystokinin. The level of the gastrointestinal hormone may be measured by the biometric sensor and stored in the memory 104, and the average level may be calculated by the processor 106 by using data stored in the memory 104. The answer list information 110 is provided for the illustrative purpose according to an embodiment, and the processor 106 may generate at least one of answer list information 110 by combining data included in the bio-information with data included in the function execution information.

Referring to FIGS. 6 and 7, the processor 106 may display automatically generated responses (e.g., any one piece of information) selected from at least one piece of answer list information 110 by a user input, on the input window 112 or the chat window 114. The input window 112 may be a window into which the user inputs a character, and the chat window 114 may be a window on which the string 202 is displayed.

Referring to FIGS. 5 and 6, if the user selects information i) from among the possible selectable responses “I have many business meetings these days, so tired.”, “I am free these days, so I am in good condition.”, and “I am not feeling well due to frequent drinking.”, ‘information i)’ may be indicated as a reply (e.g., answer information). If information i) becomes the chosen reply (e.g., the answer information), the processor 106 may set up such that information i) is displayed on the input window 112. Referring to FIGS. 5 and 7, according to another embodiment, as soon as the user selects information i), the processor 106 may display information i) on the chat window 114. According to an embodiment of the present disclosure, the user selects any one piece of information from among one or more pieces of answer list information 110, thereby easily inputting the answer information to the string 202 into the electronic device 100.

FIG. 8 illustrates generation of answer list information 110 related to foods, according to an embodiment. Hereinafter, the generation of the answer list information 110 related to the foods according to one embodiment of the present disclosure will be described with reference to FIG. 2 and FIG. 8.

Referring to FIG. 8, when the string 202 is received, the processor 106 may extract a character related to food from the string 202. For example, if a character list related to food is stored in the memory 104 inside the electronic device 100, and if the character related to food is included in the received string 202, the processor 106 may extract the character. Referring to FIG. 8, “pizza” may be stored as a character related to food in the memory 104. Accordingly, if the string 202 of “Why don't we eat a pizza?” is received, the processor 106 may extract “pizza” which is a word or string related to food.

If the character related to food is extracted, bio-information related to calorie and function execution information related to the bio-information may be extracted. For example, the relation among the character, which is related to food, the bio-information related to the calorie, and the function execution information related to the bio-information may be stored in the memory 104. Accordingly, if the character related to the foods is extracted, the processor 106 may extract the bio-information related to the calorie corresponding to the character from the memory 104. In addition, the processor 106 may extract the function execution information corresponding to the bio-information related to the calorie from the memory 104.

If the bio-information and the function execution information are extracted, the processor 106 may generate at least one piece of answer list information 110 based on the extracted bio-information and the extracted function execution information. For example, if “pizza”, which is the character related to the foods, is extracted as illustrated in FIG. 8, the processor 106 may determine calories, which are taken for one week or for one month, from the bio-information. Since a weight is closely related to the taken calories, the processor 106 may determine weights accumulated for one week or for one month. If the bio-information is extracted, the processor 106 may determine schedules, which are performed for one week or for one month, from the function execution information.

If it is determined that a lunch date has been made and taken calories represent a high value (e.g., greater than predetermined threshold) before the string 202 is received, the processor 106 may generate the answer list information 110 of “No, I had a lunch in the restaurant and I am full”. In addition, if it is determined that the record of eating a pizza is absent and the weight is lower than an average weight, the processor 106 may generate the answer list information 110 of “I have never been a pizza recently and want to eat the pizza.” In addition, if it is determined that a schedule is absent and calories are not taken, the processor 106 may generate the answer list information 110 of “I have no appointment today, so let's eat a pizza.” The answer list information 110 is provided for the illustrative purpose according to an embodiment, and the processor 106 may generate at least one piece of answer list information 110 by combining data included in the bio-information with data included in the function execution information.

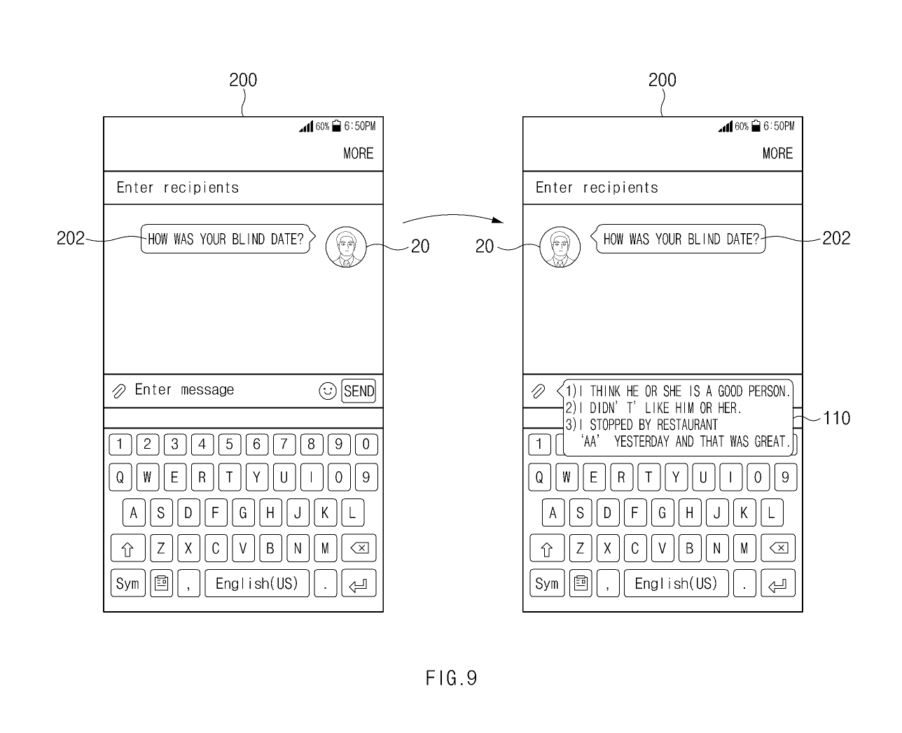

FIG. 9 illustrates generation of answer list information 110 related to a schedule, according to an embodiment.

Referring to FIG. 9, the electronic device 100 may further include a position sensor which senses the position of a user, and the position sensor may be a global positioning system (GPS). If the position sensor senses the position of the user, the memory 104 may store position information of the user as function execution information. Hereinafter, the generation of the answer list information 110 related to the schedule will be described with reference to FIG. 2 and FIG. 9.

Referring to FIG. 9, if the string 202 is received, the processor 106 may extract a character related to the schedule from the string 202. For example, if a character list related to the schedule is stored in the memory 104 and if the character related to the schedule is present in the received string 202, the processor 106 may extract the character. Referring to FIG. 9, the characters of “blind date”, which are related to a user's schedule, and may be stored in the memory 104. Accordingly, if the string 202 of “How was your blind date?” is received, the processor 106 may extract “blind date” which are characters related to and indicating a user's schedule.

If the character related to the schedule is extracted, bio-information obtained during the schedule and function execution information related to positions during the schedule may be extracted. For example, the relation among the character, which is related to the schedule, the bio-information formed during the schedule, and the function execution information related to the positions during the schedule may be stored in the memory 104. Accordingly, if the character related to the schedule is extracted, the processor 106 may extract the bio-information, which is obtained during the schedule, from the memory 104. In addition, the processor 106 may extract the function execution information related to the positions during the schedule from the memory 104.

If the bio-information and the function execution information are extracted, the processor 106 may generate answer list information based on the extracted bio-information and the extracted function execution information. For example, if “blind date”, which are characters related to the schedule, are extracted as illustrated in FIG. 9, the processor 106 may determine heart rates, blood pressure, and the like which were measured contemporaneously when the blind date occurred (as indicated by the user's past schedules) from the bio-information. If the bio-information is extracted, the processor 106 may determine a position (or location), in which the blind date occurred, and further information such as a travel distance, which may be obtained from the function execution information.

If it is determined that the heart rates and the blood pressure represent high values (e.g., by comparison against a threshold) and the travel distance (e.g., moving distance) was short during the blind date, the processor 106 may generate the answer list information 110 of “I think he or she is a good person.” If it is determined that the heart rates and the blood pressure represent low values and the moving distance is long during the blind date, the processor 106 may generate the answer list information 110 of “I didn't' like him or her.” If it is determined that the heart rates and the blood pressure represent high values (e.g., greater than predetermined threshold) and a record of stopping by restaurant AA is present, the processor 106 may generate the answer list information 110 of “I stopped by restaurant AA and that was great” The above-described answer list information 110 is provided for the illustrative purpose according to an embodiment, and the processor 106 may generate at least one piece of answer list information 110 by combining data included in the bio-information with data included in the function execution information.

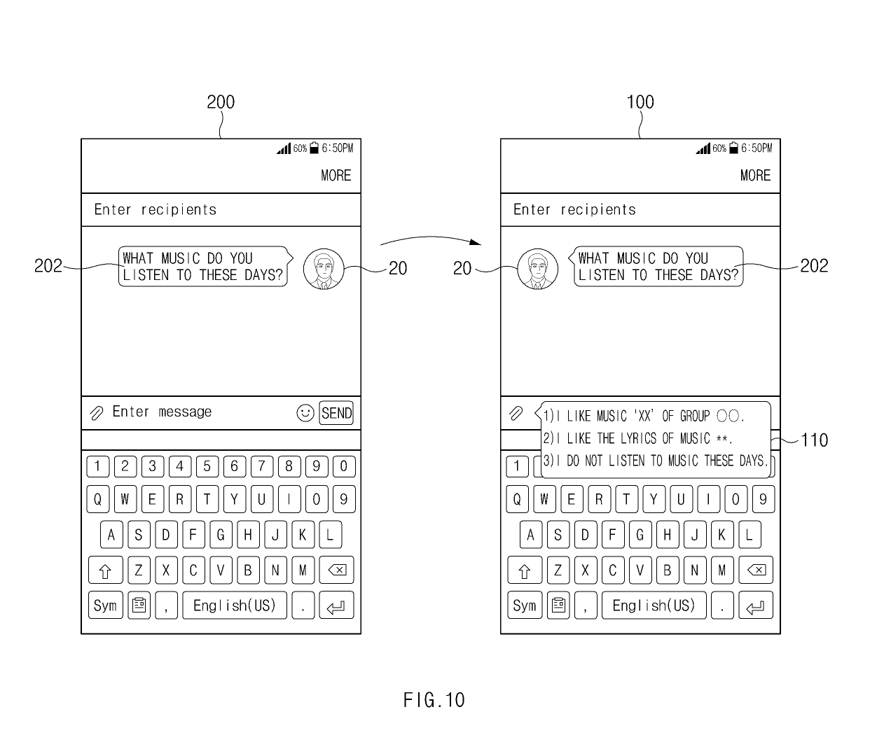

FIG. 10 illustrates the generation of answer list information 110 related to a user interest, according to an embodiment. Hereinafter, the generation of the answer list information 110 related to the user interest according to one embodiment of the present disclosure will be described with reference to FIGS. 2, 4, and 10.

Referring to FIG. 4, the processor 106 may generate a characteristic related to the user interest based on search information (e.g., a specific category of user interest), which is repeatedly input by the user, in the function execution information. For example, if the user repeatedly searches for music “XX” of group “∘∘” through the electronic device 100, then “music XX of group ∘∘” may be designated as search information, and the characteristic (or specific category) related to the user interest may be “music.” For example, if the user repeatedly searches for film “ABCD” through the electronic device 100, “ABCD” may be designated as the search information, and the characteristic related to (or category of) the user interest may be indicated as a “film.”

Referring to FIG. 10, if the string 202 is received, the processor 106 may extract a character related to the user interest from the string 202. For example, if the string 202 of “What music do you listen to these days?” is received, the processor 106 may extract “music” which is a characteristic related to the user interest (e.g., a specific category of user interest).

Accordingly, if the character related to the user interest is extracted, the processor 106 may extract the bio-information related to the heart rate, from the memory 104. If the bio-information is extracted, the processor 106 may generate at least one possible response (e.g., a piece of answer list information 110) based on the extracted bio-information and the extracted search information. For example, if “music” (the character related to the user interest) is extracted, as illustrated in FIG. 10, the processor 106 may determine heart rate and blood pressure from the bio-information, which was accumulated for a time period such as a week or a month. Then, the processor 106 may determine music that was searched for by the user during the same week or month, which may be retrieved from the function execution information.

If it is determined that the heart rates and the blood pressure represent high values (e.g., greater than predetermined threshold) and music XX of group ∘∘ is frequently searched for, the processor 106 may generate answer list information 110 of “I like music XX of group ∘∘.” If it is determined that the heart rates and the blood pressure represent high values (e.g., greater than predetermined threshold) and the lyrics of music “*” is frequently searched for, the processor 106 may generate answer list information 110 of “I like the lyrics of music **.” In addition, if it is determined that no music has ever been searched for, the processor 106 may generate answer list information 110 of “I do not listen to music these days.” The above-described answer list information 110 is provided for the illustrative purpose according to an embodiment, and the processor 106 may generate at least one piece of answer list information 110 by combining data included in the bio-information with data included in the function execution information.

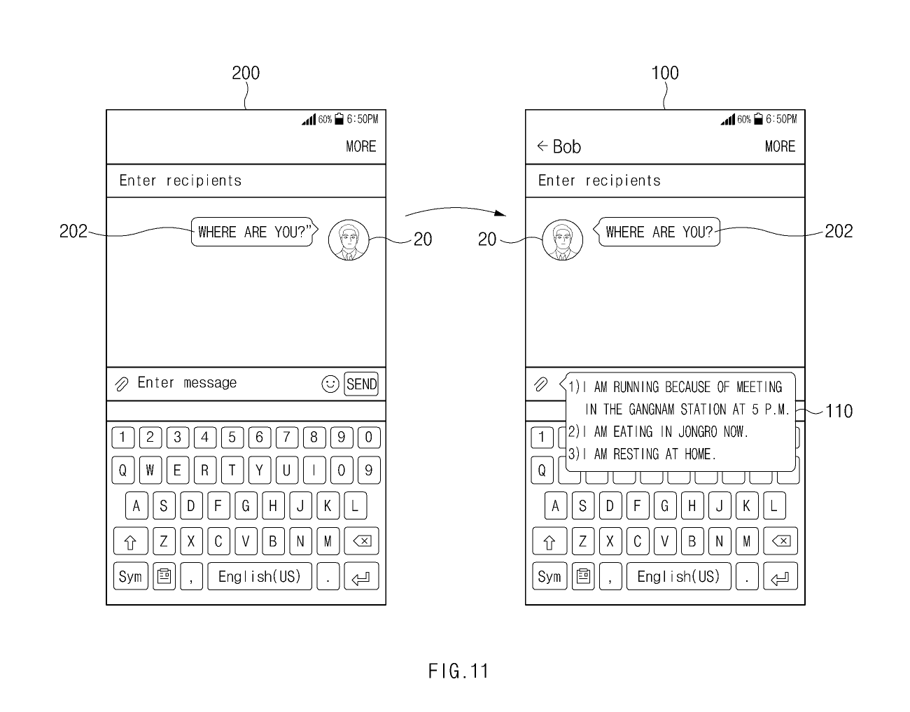

FIG. 11 illustrates generation of answer list information 110 related to a position, according to an embodiment. Hereinafter, generation of the answer list information 110 related to the position according to one embodiment of the present disclosure will be described with reference to FIG. 2 and FIG. 11.

Referring to FIG. 11, the electronic device 100 may further include a position sensor which senses the position of a user, and the position sensor may be a global positioning system (GPS). If the position sensor senses the position of the user, the memory 104 may store position information of the user as function execution information.

Meanwhile, if the string 202 is received, the processor 106 may extract a character related to the position from the string 202. Accordingly, if the string 202 of “Where are you?” is received, the processor 106 may extract “where” which is a character related to a user location (e.g., the position).

If the character is received, the processor 106 may extracts bio-information related to the heart rate from the memory 104 and may generate at least one response (e.g., a piece of answer list information 110) based on the extracted bio-information and the position information of the user. For example, if “where”, which is the character related to the position, is extracted as illustrated in FIG. 11, the processor 106 may determine a heart rate, blood pressure, oxygen saturation, taken calories, or the like in the present state from bio-information. If the bio-information is extracted, the processor 106 may determine a position, a moving distance, a moving route or the like, from the function execution information.

If it is determined that the heart rate and the blood pressure represent high values (e.g., greater than a predetermined threshold), a meeting is planned, and a present position is ‘Gangnam’, the processor 106 may generate answer list information 110 of “I am running because of meeting in the Gangnam station at 5 p.m.” In addition, if it is determined that taken calories are increased and the position is Jongro, the processor 106 may generate answer list information 110 of “I am eating in Jongro now.” In addition, if it is determined that the oxygen saturation represents a high level (e.g., greater than predetermined threshold) and the position is home, the processor 106 may generate answer list information 110 of “I am resting at home.” The above-described answer list information 110 is provided for the illustrative purpose according to an embodiment, and the processor 106 may generate at least one piece of answer list information 110 by combining data included in the bio-information with data included in the function execution information.

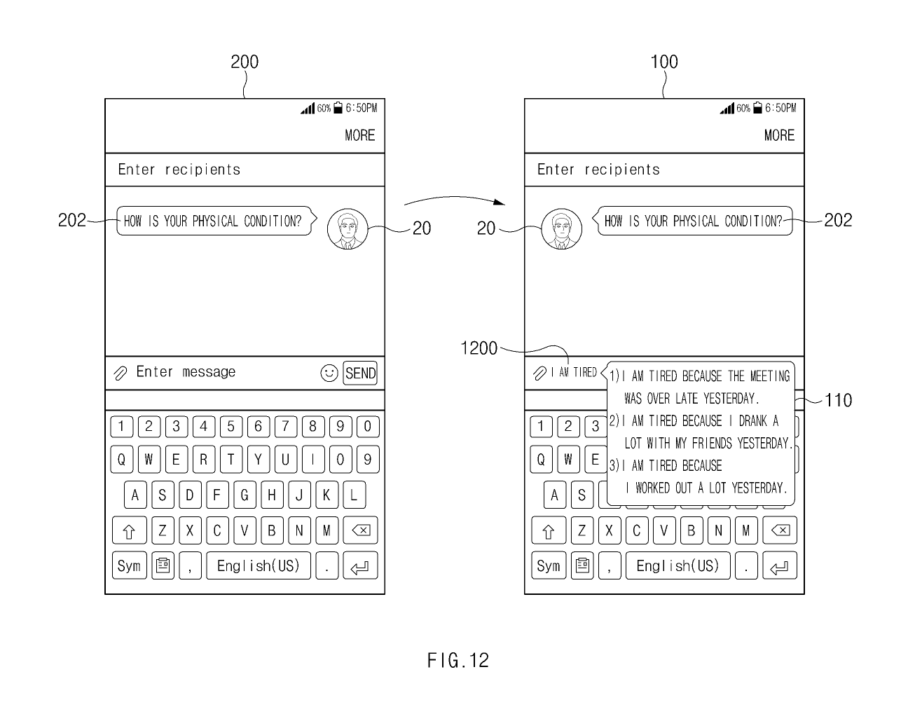

FIG. 12 illustrates the generation of answer list information based on user input information (e.g., historical information related to biometrics or electronic device functions that were previously input by a user), according to an embodiment. Hereinafter, the generation of answer list information 110 related to the user input information according to one embodiment of the present disclosure will be described with reference to FIG. 2 and FIG. 12.

Referring to FIG. 12, the electronic device 100 may receive user input information 1200 corresponding to the string 202 through the input window 112 displayed on the display 108. The user input information 1200 may be information which is personally input by a user corresponding to the string 202 and may include bio-information and function execution information. After the user input information 1200 is input, the processor 106 may generate answer list information 110 based on bio-information related to the user input information 1200 and function execution information related to the user input information 1200.

For example, if the user input information 1200 of “I am tired” is input, the connection between the user input information 1200 and the bio-information may be stored in the memory 104 such that bio-information, such as a sleep record, blood pressure, a heart rate, a lactate level, or the like, may be searched for. In addition, if the user input information 1200 of “I am tired” is input, the connection relation between the user input information 1200 and the function execution information may be stored in the memory 104 such that function execution information related to the schedule may be searched for.

For example, if the user input information 1200 of “I am tired” is input as illustrated in FIG. 12, the processor 106 may determine bio-information, such as a sleep record, blood pressure, a heart rate, a lactate level, or the like, and function execution information such as a schedule, a moving distance, or the like. If it is determined that a sleep time is short and a business meeting was present yesterday, the processor 106 may generate the answer list information 110 of “I am tired because the meeting was over late yesterday.” If it is determined that a sleep time is short and a meeting was present with friends, the processor 106 may generate answer list information 110 of “I am tired because I drank a lot with my friends yesterday.” If it is determined that the lactate level represents a high level (e.g., greater than predetermined threshold) and a workout record is present, the processor 106 may generate the answer list information 110 of “I am tired because I worked out a lot yesterday.” The above-described answer list information 110 is provided for the illustrative purpose according to an embodiment, and the processor 106 may generate at least one of answer list information 110 by combining data included in the bio-information with data included in the function execution information.

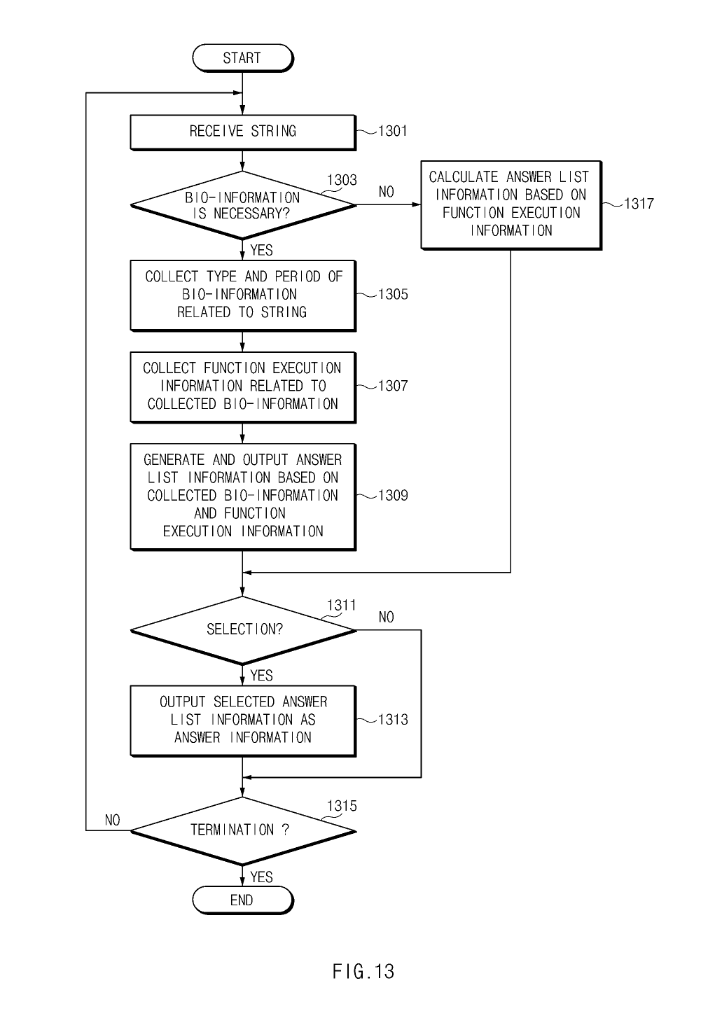

FIG. 13 is a flowchart illustrating the generation of answer list information 110, according to an embodiment. Hereinafter, the generation of the answer list information 110 according to another embodiment of the present disclosure will be described with reference to FIGS. 2 and 13.

Referring to FIG. 13, the memory 104 may store bio-information, and the bio-information may be substantially identical to the bio-information described with reference to FIG. 3. After the memory 104 stores the bio-information, the communication interface 102 may receive the string 202 from the terminal 200 in operation 1301 and may provide the string 202 to the processor 106. If the string 202 is received, the processor 106 may determine whether a character related to, corresponding to or triggering retrieval the bio-information is included in the string 202. For example, if the string 202 of “How is your physical condition?” is received, and if a character of ‘condition’ is stored as a character related to the bio-information in the memory 104, the processor 106 may determine the character related to the bio-information as being included in the string 202. Other examples have been shown above, as related to other categories such as a time period (“these days”), food (“pizza”), scheduled events (“blind date”), user interests (“music”), user location (“where”), and user condition (“physical condition”), etc.

In operation 1303, the processor 106 may determine whether bio-information is necessary to generate at least one piece of answer list information 110 corresponding to the string 202. In other words, if the character related to the bio-information is included in the string 202, the processor 106 may determine that bio-information is necessary to generate the answer list information 110. For example, in one embodiment, a number of keywords may be stored. When a received message is parsed and found to include one of the keywords, such as relating to time period (“these days”), food (“pizza”), scheduled events (“blind date”), user interests (“music”), user location (“where”), and user condition (“physical condition”), etc., then it may be determined that bio-information is necessary.

If the processor 106 determines that the bio-information is necessary, the processor 106 may collect bio-information related to at least a portion of the string 202 from the memory in operation 1305 and may collect function execution information in operation 1307. In operation 1307, the memory 104 may store function execution information which is related to a designated period and the processor 106 may collect the function execution information from the memory 104. If the bio-information and the function execution information are collected, the processor 106 may generate the possible responses (e.g., the answer list information 110 based on the bio-information and the function execution information in operation 1309. The possible responses may be generated by, for example, storing a plurality of incomplete phrases as templates, and inserting the requisite information into the phrases (or keywords, biometric related words, or other related words by pre-association) based on the retrieved bio-information and the keywords. Further, certain incomplete phrases may be pre-associated with certain keywords or bio-information in order to ascertain which template should be used to generate a response. Certain biometric markers may be pre-associated with value-words, such as “many,” “free,” “tired,” “good,” “not feeling well,” “frequent,” “drinking,” “fine,” etc., which may then be selected and used to complete the template responses.

According to an embodiment, the processor 106 may generate a character related to the user interest based on search information, which is repeatedly input by the user, of the function execution information. If a character related to the user interest is included in the string 202, the processor 106 may generate answer list information 110 based on the bio-information and the search information related to at least a portion of the string 202. A manner of generating the answer list information 110 related to the user interest may be substantially identical to the method described with reference to FIG. 10.

According to an embodiment, if the character related to a position is extracted from the string 202, the processor 106 may generate answer list information 110 based on bio-information related to the character and the position information of a user. A manner of generating the answer list information 110 related to the position by the processor 106 may be substantially identical to the method described with reference to FIG. 11.

According to one embodiment, if the processor 106 determines that bio-information is necessary to generate the answer list information, the processor 106 may be configured to measure bio-information through the biometric sensor 300 in real time. In this case, the biometric sensor 300 may be substantially identical to the biometric sensor 300 described with reference to FIG. 1. If the biometric sensor 300 measures the bio-information, the processor 106 may generate the answer list information 110 based on the obtained bio-information and the function execution information stored in the memory 104.

After the answer list information 110 is generated, the processor 106 may display any one pieces of answer list information selected by a user input on the input window 112, into which the user inputs a character, in operation 1311 and operation 1313. In addition, the processor 106 may display any one piece information, which is selected from answer list information 110 by the user input, on the chat window 114 on which the string 202 is displayed. A manner of displaying answer information on the input window 112 and a manner of displaying answer information on the chat window 114 may be substantially identical to manners described with reference to FIGS. 6 and 7. Subsequently, the process may end in operation 1315, unless further messages/texts/strings are received.

Meanwhile, in operation 1303, if a character related to bio-information is not included in the string 202, the processor 106 may not determine bio-information as being necessary to generate answer list information 110. If it is determined that the bio-information is unnecessary, the processor 106 may calculate answer list information 110 based on the function execution information in operation 1317. In operation 1317, the memory 104 may store function execution information and the processor 106 may collect the function execution information from the memory 104. After the function execution information is collected, the processor 106 may calculate answer list information 110 based on the function execution information in operation 1317.

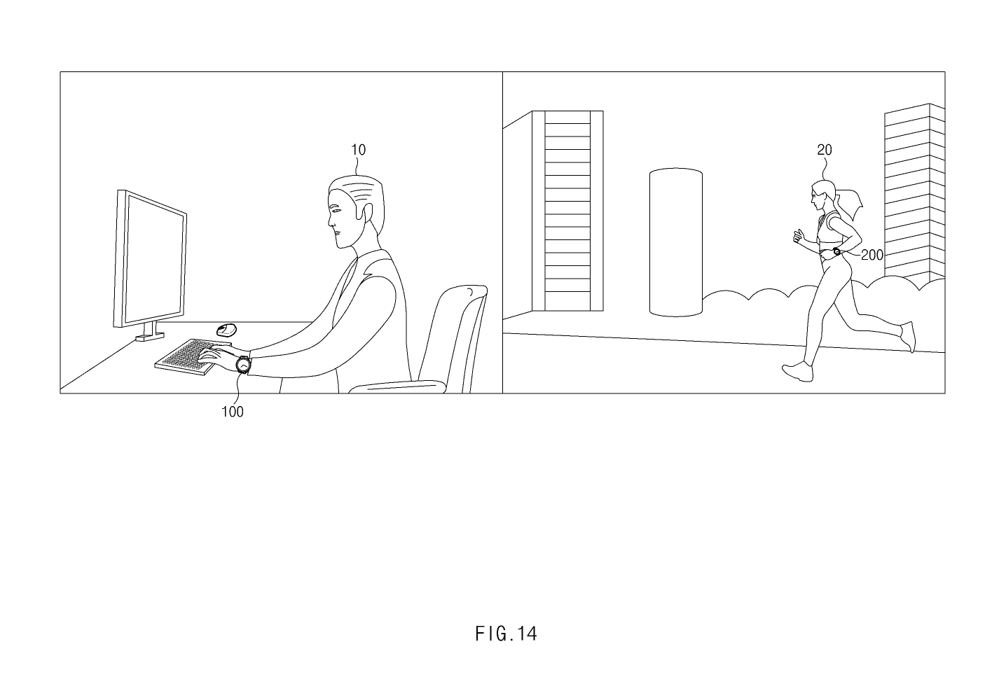

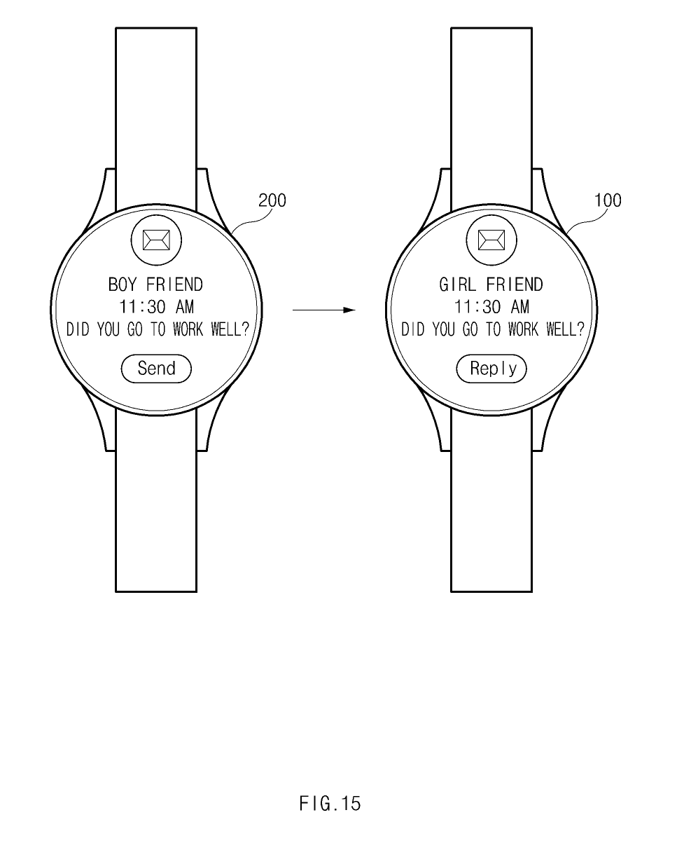

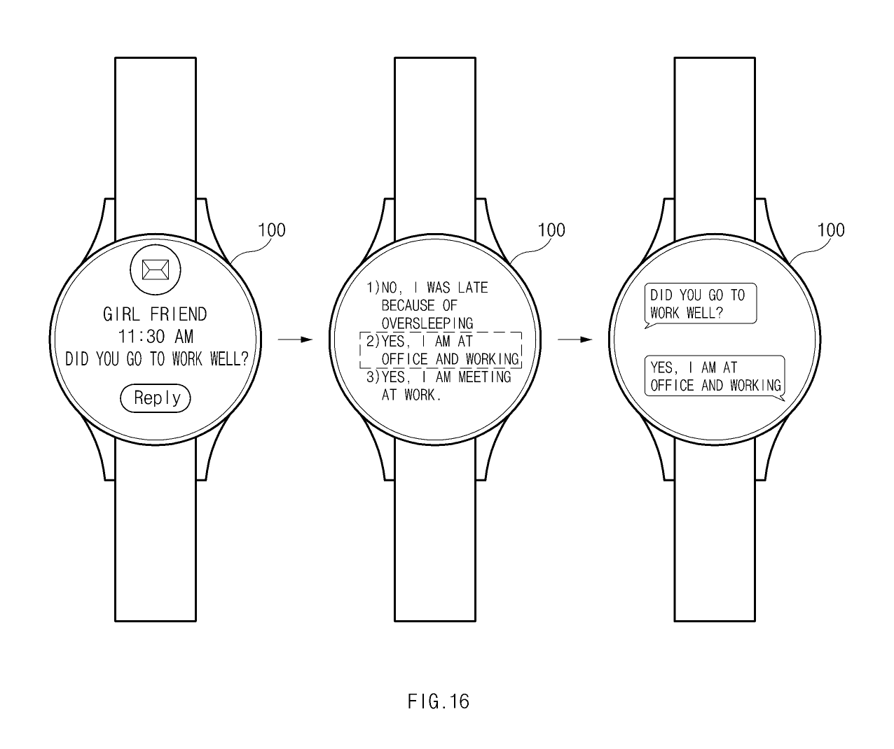

FIG. 14 illustrates one example of an operating environment of an electronic device, according to another embodiment. FIG. 15 illustrates an electronic device receiving a string from a terminal, according to another embodiment. FIG. 16 illustrates the output of answer list information and answer information through an electronic device, according to another embodiment. Hereinafter, an electronic device according to another embodiment of the present disclosure will be described with reference to FIGS. 14 to 16.

Referring to FIG. 14, the electronic device 100 and the terminal 200 may implemented as a smartwatch, and thus may make contact with bodies of a user 10 and a terminal user 20. The form of the electronic device 100 and the terminal 200 is not limited to smartwatches, but may be implemented in other formats, such as head bands or arm bands. In addition, the electronic device 100 and the terminal 200 may be the same type of device, or different types of devices.

Referring to FIGS. 14 and 15, the terminal user 20 may input a string of “Did you go to work well?” into the terminal 200. After the string is input, when the terminal user 20 touches a displayed button indicating “send”, the string may be transmitted to the electronic device 100. If the string is transmitted to the electronic device 100, the electronic device 100 may display a button indicating “reply” along with the message (e.g., the string) on an output screen, allowing the user 10 to reply to the message.

Referring to FIG. 16, the user 10 may select the button “reply” to answer to the message. If the user 10 selects (e.g., touches) the “reply” button, the electronic device 100 may output a number of possible responses as an answer list via the output screen. Procedures of generating the answer list information may correspond to procedures described with reference to FIGS. 1 to 13.

If the user 10 selects any one piece of answer list information, the selected answer list information may be output through the electronic device 100. In FIG. 16, if the user selects “Yes, I am at office and working.”, among “No, I was late because of oversleeping.”, “Yes, I am at office and working.”, and “Yes, I am meeting at work.”, the selected answer list information may be output.

According to another embodiment of the present disclosure, the answer list information corresponding to the received string is provided. Accordingly, the user may easily input answer information even in the electronic device having a smaller input window.

At least a part of an apparatus (e.g., modules or functions thereof) or a method (e.g., operations) according to various embodiments may be, for example, implemented by instructions stored in a computer-readable storage media in the form of a program module. The instruction, when executed by a processor, may cause the one or more processors to perform a function corresponding to the instruction. The computer-readable storage media, for example, may be the memory.

In addition, according to an embodiment disclosed in the present disclosure, a non-transitory storage medium for storing computer-readable instructions that, when executed by an electronic device, may cause the electronic device to store bio-information, which is obtained by accumulating body status information related to a body status of a user for a designated period, and function execution information related to a function of the electronic device; receiving a string, to extract bio-information related to at least a portion of the string from the memory, to extract function execution information related to the extracted bio-information from the memory, generating at least one piece of answer list information corresponding to the string based on the extracted bio-information and the extracted function execution information, and to display the answer list information.

A computer-readable recording medium may include a hard disk, a floppy disk, a magnetic media (e.g., a magnetic tape), an optical media (e.g., a compact disc read only memory (CD-ROM) and a digital versatile disc (DVD), a magneto-optical media (e.g., a floptical disk)), and hardware devices (e.g., a read only memory (ROM), a random access memory (RAM), or a flash memory). Also, a program instruction may include not only a mechanical code such as things generated by a compiler but also a high-level language code executable on a computer using an interpreter. The above hardware unit may be configured to operate via one or more software modules for performing an operation of various embodiments of the present disclosure, and vice versa.

A module or a program module according to various embodiments may include at least one of the above elements, or a part of the above elements may be omitted, or additional other elements may be further included. Operations performed by a module, a program module, or other elements according to various embodiments may be executed sequentially, in parallel, repeatedly, or in a heuristic method. In addition, some operations may be executed in different sequences or may be omitted. Alternatively, other operations may be added.

According to embodiments disclosed in the present disclosure, the answer list information is provided corresponding to the string based on the status of the user, thereby easily inputting the answer information into an input window. In addition, the present disclosure may directly or indirectly produce various effects.

While the present disclosure has been illustrated and described with reference to various embodiments thereof, it will be understood by those skilled in the art that various changes in form and details may be made therein without departing from the present disclosure as defined by the appended claims and their equivalents.

The control unit or processor may include a microprocessor or any suitable type of processing circuitry, such as one or more general-purpose processors (e.g., ARM-based processors), a Digital Signal Processor (DSP), a Programmable Logic Device (PLD), an Application-Specific Integrated Circuit (ASIC), a Field-Programmable Gate Array (FPGA), a Graphical Processing Unit (GPU), a video card controller, etc. In addition, it would be recognized that when a general purpose computer accesses code for implementing the processing shown herein, the execution of the code transforms the general purpose computer into a special purpose computer for executing the processing shown herein. Any of the functions and steps provided in the Figures may be implemented in hardware, software or a combination of both and may be performed in whole or in part within the programmed instructions of a computer. In addition, an artisan understands and appreciates that a “processor” or “microprocessor” may be hardware in the claimed disclosure. Under the broadest reasonable interpretation, the appended claims are statutory subject matter in compliance with 35 U.S.C. §101.

In addition, it would be recognized that when a general purpose computer accesses code for implementing the processing shown herein, the execution of the code transforms the general purpose computer into a special purpose computer for executing the processing shown herein. Any of the functions and steps provided in the Figures may be implemented in hardware, or a combination hardware configured with machine executable code and may be performed in whole or in part within the programmed instructions of a computer. No claim element herein is to be construed under the provisions of 35 U.S.C. 112, sixth paragraph, unless the element is expressly recited using the phrase “means for”.

The terms “unit” or “module” referred to herein is to be understood as comprising hardware such as a processor or microprocessor configured for a certain desired functionality, or a non-transitory medium comprising machine executable code, in accordance with statutory subject matter under 35 U.S.C. §101 and does not constitute software per se.

In addition, an artisan understands and appreciates that a “processor” or “microprocessor” constitute hardware in the claimed invention. Under the broadest reasonable interpretation, the appended claims constitute statutory subject matter in compliance with 35 U.S.C. §101.

The functions and process steps herein may be performed automatically or wholly or partially in response to user command. An activity (including a step) performed automatically is performed in response to executable instruction or device operation without user direct initiation of the activity.

What is claimed is:

1. An electronic device comprising:
a display;
a memory configured to store bio-information of a user collected over a designated time period, and function information for executable functions of the electronic device; and
a processor configured to:
when a textual message includes a predesignated keyword is received, retrieve particular bio-information corresponding to the keyword from the stored bio-information, and a particular function information corresponding to the retrieved bio-information from the stored function information;
generate one or more textual responses to the received textual message based on the retrieved particular bio-information and the particular function information; and
control the display to display an answer list including the generated one or more textual responses.

2. The electronic device of claim 1, wherein when the keyword indicates a particular time period, the particular bio-information and the particular function information are retrieved from the memory based on correspondence to the particular time period.

3. The electronic device of claim 1, wherein when the keyword indicates foods, the particular bio-information and the particular function information are retrieved from the memory based on correspondence to caloric biometric information.

4. The electronic device of claim 1, further comprising:
a location sensor configured to detect a location of the electronic device,
wherein the function information for executable functions of the electronic device includes historical location information of the electronic device within the designated time period, and
wherein when the keyword indicates an event recorded on a scheduler stored in the electronic device, the retrieved particular bio-information corresponds to bio-information stored during the event and the retrieved particular function information are retrieved from the memory based on correspondence to the location of the electronic device during the event.

5. The electronic device of claim 1, wherein the processor is configured to:
generate a keyword related to an interest of the user based on search information, which is repeatedly input by the user, of the function information for executable functions of the electronic device.

6. The electronic device of claim 5, wherein the processor is configured to:
generate one or more textual responses based on bio-information related to a heart rate and the search information if the keyword related to an interest of the user is retrieved from the received textual message.

7. The electronic device of claim 1, further comprising:
a location sensor configured to detect a location of the electronic device,
wherein the function information for executable functions of the electronic device includes historical location information of the electronic device within the designated time period, and
when the keyword corresponds to a geographic location of the user, the processor is further configured to generate the one or more textual responses based on bio-information related to a heart rate and at least a portion of the historical location information.

8. The electronic device of claim 1, wherein the processor is configured to:
generate one or more textual responses based on bio-information related to user input and function information related to the user input, if the user input corresponding to the keyword is input through an input window displayed on the display.

9. The electronic device of claim 1, wherein the processor is configured to:
in response to detecting a selection of a response from among the generated one or more textual responses of the displayed answer list, control the display to display the selected response into a field for composing replies to the received textual message.

10. The electronic device of claim 1, further comprising:
a communication interface configured to receive textual message as transmitted from an external terminal.

11. An electronic device comprising:
a display;
a memory configured to store bio-information of a user recorded over a designated time period; and
a processor configured to:
detect whether a keyword is included in a received textual message,
if the keyword is included, retrieve particular bio-information from the stored bio-information corresponding to the keyword, and generate one or more textual responses to the received textual message based on the retrieved particular bio-information, and
control the display to display an answer list including the generated one or more textual responses.

12. The electronic device of claim 11, wherein the memory stores function information for executable functions of the electronic device,
wherein the processor is configured to:
if the keyword is included in the received textual message, retrieve particular function information corresponding to the keyword from the function information, and generate the one or more textual responses based on the retrieved particular bio-information and the retrieved particular function information.

13. The electronic device of claim 12, further comprising:
a location sensor configured to detect a location of the electronic device,
wherein the function information for executable functions of the electronic device includes historical location information of the electronic device within the designated time period, and
wherein when the keyword indicates an event recorded on a scheduler stored in the electronic device, the retrieved particular bio-information corresponds to bio-information stored during the event and the retrieved particular function information are retrieved from the memory based on correspondence to the location of the electronic device during the event.

14. The electronic device of claim 12, wherein the processor is configured to:
generate a keyword related to an interest of the user based on search information, which is repeatedly input by the user, of the function information for executable functions of the electronic device.

15. The electronic device of claim 14, wherein the processor is configured to:
generate one or more textual responses based on bio-information related to a heart rate and the search information if the keyword related to an interest of the user is retrieved from the received textual message.

16. The electronic device of claim 11, wherein the processor is configured to:
determine that at least a portion of the stored bio-information is required to generate the one or more textual responses in response to detecting that the keyword related to the portion of the stored bio-information is included in the received textual message.

17. The electronic device of claim 11, wherein the memory stores function information for executable functions of the electronic device,
wherein the processor is configured to:
determine that the stored bio-information is unrequired to generate the one or more textual responses in response to detecting the received textual message does not include the keyword,
wherein one or more textual responses are generated based on at least a portion of the stored function information.

18. The electronic device of claim 11, wherein the processor is configured to:
in response to detecting a selection of a response from among the generated one or more textual responses of the displayed answer list, controlling the display to display the selected response in a field for composing replies to the received textual message.

19. The electronic device of claim 11, further comprising:
a communication interface configured to receive the textual message from a terminal and to provide the textual message to the processor.

20. A non-transitory storage medium storing computer-readable instructions that, when executed by an electronic device, cause the electronic device to:
store bio-information of a user for a designated time period, and function information for executable functions of the electronic device,
when a textual message includes a predesignated keyword is received, retrieve particular bio-information corresponding to the keyword from the stored bio-information, and a particular function information corresponding to the retrieved bio-information from the stored function information,
generate one or more textual responses to the received textual message based on the retrieved particular bio-information and the particular function information, and
control a display to display an answer list including the generated one or more textual responses.
